(19) United States
(12) Patent Application Publication
(10) Pub. No.: US 2020/0362380 A1
(43) Pub. Date: Nov. 19, 2020

(54) Novel Biomolecule and Methods of Manufacturing Thereof

(71) Applicant: David Abecassis, Huntington Station, NY (US)

(72) Inventor: David Abecassis, Huntington Station, NY (US)

(21) Appl. No.: 16/244,338

(22) Filed: Jan. 10, 2019

Related U.S. Application Data
(60) Provisional application No. 62/615,542, filed on Jan. 10, 2018.

(51) Int. Cl. C12P 19/26

(52) U.S. Cl. CPC C12P 19/26

(57) ABSTRACT

A new previously undiscovered, undocumented biomolecule, 3-4 deoxy-glucosamine can be produced in concentrated form using a specific microbial process, and by synthesis. It can be produced in both monomer, and dimonomer forms.

This application claims the benefit of provisional filing 626155442 as a priority for this application.

### FIELD OF THE INVENTION

The field of the invention is biochemical engineering and industrial microbiology.

### BACKGROUND OF THE INVENTION

Biomolecules and media for microbial growth and selective cultivation are widely known in the public domain. The modern field of microbiology uses selective media to isolate and culture microorganisms. The inventor has discovered a novel, previously unknown bacteria species which produces a novel, unrecorded biomolecule from the growth media. The invention described herein is a method for obtaining pure concentrates of the biomolecule discovered. Shrimp and crab meal are in the public domain as commodities used for a variety of agricultural, food, and aqua-culture as well as recreational fishing uses.

In U.S. Pat. No. 4,997,469 Moore describes the use of chemically treated shrimp meal in an odor reduced nitrogen source for animal feed and fertilizer compositions.

In another example of the prior art in U.S. Pat. No. 7,208,184, Chen describes the use of shrimp and crab meal in compositions for fish bait.

Another example of the prior art in U.S. Pat. No. 5,618,574 Bunch describes their use in fish food compositions.

In U.S. Pat. No. 9,687,449, Harel describes the use of shrimp meal as part of a nutraceutical containing fish food.

The food and agriculture uses which apply to humans and livestock are regulated by the FDA, and thus subject to microbial testing. The fact that this bacteria was previously unknown to science along with it's microbial products is due to the fact that the use of these materials did not bloom sufficient biomass to detect and identify the bacteria attests to the unique conditions required to bloom it into a monoculture. In addition, many food manufacturing techniques sterilize the material destroying the organism. Analytical tests using microbiological identification are mostly geared towards identifying pathogenic organisms.

These are the primary reasons why the subject matter at the foundation of the invention is new science, and currently only revealed to the examiner.

Protein and DNA assays have failed to identify the bacteria using the largest databases available to science. This confirms the bacteria is a cryptobacteria and a novel microbiological discovery by the inventor

It's microbial products were also undetected until the inventor carried out detailed analytical testing of the products using GC/mass spectrometer.

The most advanced chemical databases available to date confirm the biomolecules produced are newly discovered by the inventor as well.

The bacteria is naturally occurring, yet normally undetectable, and subsequently it's unique products went undetected as well. This is the domaine of fundamental science only and is not being claimed as an invention, but simply as a non-published scientific study which lays the foundations of the invention claimed.

### OBJECTS OF THE INVENTION

One object of the invention is to produce concentrated 3-4 deoxy-glucosamine and 3-4 deoxy-glucosamine di-monomer. Yet another object of the invention is to create a monoculture of a specific species of bacteria. Yet another object of the invention is to create 3-4 deoxy-glucosamine synthetically using chemical catalysts. Yet another object of the invention is to create 3-4 deoxy-glucosamine monomer using immobilized or dissolved enzymes. Another object of the invention is to produce the monomer 3-4 deoxy-glucosamine and it's di-monomer form in solid state fermentation on wetted media powder. Another object of the invention is to create a monoculture of the 3-4 deoxy-glucosamine producing cryptobacteria in water and in aqueous fermentation. Yet another object of the invention is to synthesize glucosamine into deoxyglucosamine in solution with chemical reducing agents.

### SUMMARY OF THE INVENTION

The invention is a method for synthesizing and selectively biosynthesizing the purified monomer and dimer of 3-4 deoxy-glucosamine using a cryptobacteria and or synthetic reduction of adjacent hydroxyls on the glucosamine molecule precursor in solution.

### DETAILED DESCRIPTION OF THE INVENTION

Shrimp and crab meal are micronized into a fine powder and fermented under aerobic conditions in pure form to selectively bloom a cryptobacteria which in turn biodegrades the meal exclusively into 3-4 deoxy-glucosamine. In another preferred embodiment, glucosamine is dissolved into a solvent with stocheometric amounts of catalyzed reducing agent and reduced to deoxy-glucosamine.

### Examples of the Invention

Crab and or shrimp meal are micronized into a fine powder. The ensuing powder is then applied to a porous surface and wetted enough to completely moisten the powder. The powder is spread out to a few millimeters of thickness to allow maximum aerobic mass exchanges. The crypto-bacteria is allowed to bloom at room temperature for 48-72 hours and does so to the exclusion of all microorganisms excepting the cryptobacteria. The cryptobacteria fully bloomed into a monoculture, biodegrades the crustacean meal and converts all the available biopolymers to only one microbial product, 3-4 deoxy-glucosamine.

The resulting monomer can be leached through the media column and recuperated in pure form after filtering out any bacteria using appropriate filtration such as diatomaceous earth or filter paper. This can also be carried out in media based agriculture both with and without soil. The monomer has a pronounced auxin effect on plants.

In another example of the invention, 5 grams of micronized shrimp or crab meal is added per gallon of tap-water, and subjected to a vortex in a fermenter under maximum oxygenation. After forty eight hours of fermentation, the cryptobacteria blooms on the meal particles and begins to biodegrade them into a solution which contains only the bacteria and the monomer and dimer of 3-4 deoxy-glucosamine and meal particles which eventually is completely converted to biomass and the desired monomer.

In another example, glucosamine is dissolved in a solvent, and treated with a reducing agent in the presence of catalysts. The 3-4 deoxy-glucosamine which results is purified out of solution by fractional crystallization.

1) The use wetted micronized shrimp meal or crab meal, to selectively bloom the cryptic, previously undiscovered bacteria, (named Streptococcus auxinotrope by the inventor), in order to generate concentrated pure 3-4 deoxy-glucosamine from a monoculture of the afore-mentioned cryptobacteria.

2) The claim in 1 where the wetting takes place in an oxygenated aqueous slurry fermentation with a solids range of one to ten grams per gallon of water and is a monoculture of the crypto-bacteria producing the 3-4 deoxy-glucosamine in an aqueous solution.

3) The claim in 1 where the micronized shrimp or crab meal particles are mixed with cellulose fibers ranging from 20 to 300 microns in length for use in wetted solid media fermentation.

4) The use of catalyst after methylation of hydroxyls in all positions excepting carbons 3 and 4, to selectively remove two adjacent hydroxyl groups from glucosamine, by simple reduction, or deoxygenation, in order to produce 3-4 deoxy-glucosamine.

5) The use of catalyst, to selectively remove two adjacent hydroxyl groups from glucosamine where the catalyst is purified deoxy-glucoseamine synthetase enzyme from a fractionation extract of cryptobacteria and subsequent gel column purification of the enzyme.
